(19) United States
(12) Patent Application Publication
(10) Pub. No.: US 2002/0155991 A1
(43) Pub. Date: Oct. 24, 2002

(54) Anti-wrinkle cosmetic or pharmaceutical compositions comprising polymers and salts thereof

(76) Inventors: Michel Philippe, Wissous (FR); Sylvie Benard, Attainville (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/086,451

(22) Filed: Mar. 4, 2002

(30) Foreign Application Priority Data
Mar. 5, 2001 (FR) 01 02979

(51) Int. Cl. A61K 38/16

(52) U.S. Cl. 514/2

(57) ABSTRACT

An anti-wrinkle composition comprising, in a physiologically acceptable medium, at least at least one material chosen from polymers and salts thereof, of formula (I), also referred to as at least one material of polyamino acid derivative type, and also to the use of this at least one material for reducing, fading out and/or smoothing out wrinkles and/or fine lines in skin. The invention also relates to the use of this compound as a tensioning agent for reducing, smoothing out and/or fading out wrinkles and/or fine lines in skin.

The present invention relates, in one aspect, to the use of at least one material chosen from polymers and salts thereof, of formula (I), as defined herein, which can be referred to as polyamino acid derivatives, in a composition, such as a cosmetic or dermatological composition. The composition can be applied to skin or mucous membranes, for example, to the face, for treating, that is to say reducing, fading out and/or smoothing out, wrinkles and/or fine lines in human skin.

In the course of the ageing process, various characteristic signs appear on the skin, which are reflected especially by a modification in the cutaneous structure and functions. The main clinical signs of ageing of the skin include the appearance of wrinkles and/or fine lines, which increase with age. Disruption of the “grain” of the skin can be observed, that is to say that the microrelief is less uniform and has an anisotropic nature.

It is known practice to treat these signs of ageing by using cosmetic or dermatological compositions containing active agents that are capable of combating ageing, such as α-hydroxy acids, β-hydroxy acids and retinoids. These active agents act on wrinkles by removing the dead cells from the skin and by accelerating the process of cell renewal. However, these active agents have the drawback of not being effective for the treatment of wrinkles until they have been applied for a certain time. Now, it is increasingly sought to obtain an immediate effect, leading rapidly to a smoothing-out of wrinkles and/or fine lines and to the disappearance, even temporary, of fatigue marks.

It has been proposed to use an aqueous dispersion of polymer particles as a skin-tensioning agent, leading to a camouflaging of wrinkles by smoothing out the skin. However, these compositions are always in aqueous form, which may lead, firstly, to bacteriological problems, and above all, secondly, to an excessively easy removal of make-up in the presence of water. Specifically, it is not possible, with this solution, to prepare a composition that has good water resistance.

The inventors have found, surprisingly and unexpectedly, that the use of polymers and salts thereof makes it possible to obtain a composition that can be applied to skin and that can improve the “camouflaging” and/or fading out of the wrinkles and/or fine lines already formed; this effect can be obtained immediately, i.e., within 3 hours.

The polymers and salts thereof used in the present invention can comprise effective tensioning agents. The expression “tensioning agent” means compounds capable of having a tensioning effect, that is to say compounds that can make the skin taut and, by this tensioning effect, smooth out skin and make the wrinkles and fine lines therein reduce or even disappear immediately.

One subject of the invention is thus an anti-wrinkle composition comprising, in a physiologically acceptable medium, at least one material chosen from polymers and salts thereof, of formula (I),

Another subject of the invention is the use of at least one material chosen from polymers and salts thereof, of formula (I), or of a cosmetic composition comprising it, to reduce, fade out and/or smooth out wrinkles and/or fine lines in skin.

Another subject of the invention is the use of at least one material chosen from polymers and salts thereof, of formula (I), for the manufacture of a pharmaceutical composition for reducing, fading out and/or smoothing out wrinkles and/or fine lines in skin.

Another subject of the invention is a tensioning agent for reducing, smoothing out and/or fading out wrinkles and/or fine lines in skin, comprising, in a physiologically acceptable medium, at least one material chosen from polymers and salts thereof, of formula (I), and uses of such materials as tensioning agents.

Another subject of the invention is a cosmetic process for treating wrinkled skin, comprising applying a composition comprising at least one material chosen from polymers and salts thereof, of formula (I), to the wrinkles and/or fine lines.

Another subject of the invention is a method for reducing, fading out and/or smoothing out wrinkles and/or fine lines in skin, comprising applying to skin a composition comprising, in a physiologically acceptable medium, at least one material chosen from polymers and salts thereof, of formula (I).

Another subject of the invention is a method of manufacturing a pharmaceutical composition for reducing, fading out and/or smoothing out wrinkles and/or fine lines in skin, comprising adding to a physiologically acceptable medium, at least one material chosen from polymers and salts thereof, of formula (I).

It has been found that the compounds according to the invention can have good skin-tensioning power. The compositions according to the invention can be easy to apply and spread easily. They make it possible to fade out the wrinkles and fine lines at the surface of the skin, immediately after application, as defined herein.

The compositions of the invention may be applied to the face and/or the neck, especially the neckline. Moreover, the synthesis of these compounds can be very fast and readily industrializable. These compounds can be prepared from well-defined amino acids, which can give oligomers and polymers with high reproducibility. This can be a great advantage when compared with natural products, for which the definition and reproducibility of the batches are very difficult to control.

The compositions according to the invention can have a light texture and can be very comfortable to wear throughout the day. They can allow a film to be obtained that has very good staying power and that is soft, supple, elastic and flexible on skin; it can follow the movements of the support on which it is deposited, without cracking and/or becoming detached. They can adhere perfectly to facial skin.

The compositions used in the present invention comprise at least one material chosen from polymers and salts thereof, of formula (I), that can be referred to as polyamino acid derivatives:

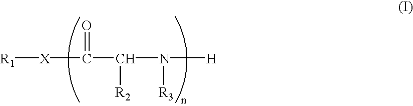

in which:

X is chosen from —O—, —S— and —NR, wherein R is chosen from hydrogen and linear and branched, saturated and unsaturated C₁-C₆ hydrocarbon-based radicals,

R₁ is chosen from:

(i) hydrogen,

(ii) linear and branched, saturated and unsaturated C₁-C₄₀ hydrocarbon-based radicals, optionally substituted with at least one group chosen from hydroxyl and —NR′R″, in which R′ and R″ are chosen, independently of each other, from hydrogen and linear and branched, saturated and unsaturated C₁-C₆ hydrocarbon-based radicals; the C₁-C₄₀ hydrocarbon-based radicals can be interrupted with at least one hetero atom chosen from N, O and Si,

(iii) a radical chosen from:

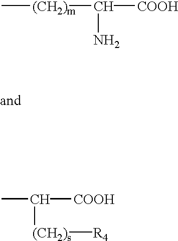

in which:

m is 1,2,3,4or5;

s is an integer between 0 and 4 inclusive;

R₄is chosen from hydrogen, —NH₂, —OH, —SH, —CHOHCH₃, —CONH₂,

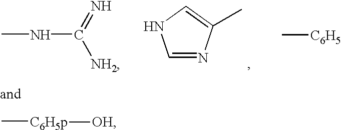

R₂ is chosen from hydrogen linear and branched, saturated and unsaturated C₁-C₈ hydrocarbon-based radicals; and radicals chosen from —CH₂OH, —CHOH—CH₃, —CH₂C₆H₅, —CH₂C₆H₄p—OH and —(CH₂)ₜ—NH₂, with t being 1, 2, 3, 4 or 5;

R₃ is chosen from hydrogen and linear and branched, saturated and unsaturated C₁-C₆ hydrocarbon-based radicals,

n is an average number of repeating units of greater than 1, such that the weight average molecular weight of the at least one material is between 200 and 200 000, inclusive, the repeating unit being either identical for the same material, in which case the material is a homopolymer, or different, R₂ and/or R₃ then taking at least one of the other meanings given for these radicals, in which case the material is a copolymer.

The at least one material may be chosen from salts of polymers of formula (I), such as mineral and organic salts that are compatible with use in cosmetics or pharmaceuticals.

In one embodiment, X is chosen from O, S and N—CH₃.

In one embodiment, R₁ is chosen from hydrogen, linear and branched, saturated and unsaturated C₁-C₂₂ hydrocarbon-based radicals, such as C₄-C₂₀ hydrocarbon-based radicals; and radicals chosen from:

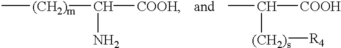

in which m, s and R₄ have the meanings given above.

In one embodiment, R₂ is chosen from hydrogen, linear and branched, saturated and unsaturated C₁-C₆ hydrocarbon-based radicals, and —CH₂C₆H₄p—OH.

In one embodiment, R₃ is chosen from hydrogen and linear and branched, saturated and unsaturated C₁-C₄ hydrocarbon-based radicals, such as methyl and ethyl.

Exemplary compounds of formula (I), in which the radical R₁ represents one of the following formulae, include:

C₁₅H₃₁—CH(OH)—CH(CH₂OH)—

C₁₀H₂₁—CH(C₈H₁₇)—CH₂—

C₁₆H₃₃—

C₈H₁₇—CH═CH—C₈H₁₆—

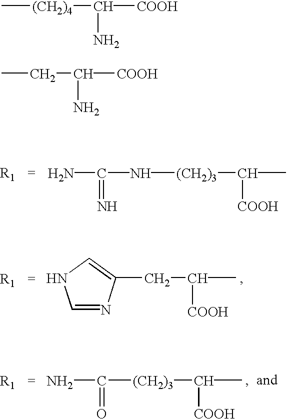

and

CH₂(OH)—CH(OH)—CH(OH)—CH(OH)—CH(OH)—CH₂—

In one embodiment, n is between 3 and 500 inclusive, and/or is such that the weight average molecular weight of the at least one material, such as a polyamino acid derivative, is between 300 and 50 000 inclusive.

The polymers and salts of formula (I) may be obtained by processes that are well known to those skilled in the art, such as by a polycondensation reaction between at least one N-carboxyanhydride of formula:

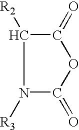

and a nucleophilic compound of formula R₁—XH in which R₁, R₂, R₃ and X have the same meanings as those given above for formula (I).

This process is described in French patent application FR 2 776 510, the disclosure of which is specifically incorporated by reference herein.

The at least one material may be used, alone or as a mixture, in an amount of from 0.001% to 30% by weight relative to the total weight of the composition, such as in an amount of from 0.01% to 15% by weight relative to the total weight of the composition.

The compounds according to the invention may accommodate various uses, especially in cosmetic or pharmaceutical compositions, which then comprise a physiologically acceptable medium, especially a cosmetically or pharmaceutically acceptable medium.

This medium, its constituents, their amount, the presentation form of the composition and the method for preparing it may be chosen by a person skilled in the art on the basis of his general knowledge, depending on the desired type of composition.

In general, this medium may be anhydrous or aqueous.

When the composition comprises an aqueous phase, the said phase may comprise water, a floral water and/or a mineral water.

The said phase may also comprise alcohols such as C₁-C₆ monoalcohols and/or polyols such as glycerol, butylene glycol, isoprene glycol, propylene glycol and polyethylene glycol.

The composition may also comprise a fatty phase, which may comprise fatty substances that are liquid at 25° C., such as volatile or non-volatile oils of animal, plant, mineral and synthetic origin; fatty substances that are solid at 25° C., such as waxes of animal, plant, mineral and synthetic origin; pasty fatty substances; gums; mixtures thereof.

The volatile oils are generally oils having, at 25° C., a saturating vapour pressure at least equal to 0.5 millibar (i.e. 50 Pa).

Among the constituents of the fatty phase that may be mentioned are:

cyclic volatile silicones containing from 3 to 8, such as from 4 to 6 silicon atoms;

cyclocopolymers such as dimethylsiloxane/methylalkylsiloxane;

linear volatile silicones comprising from 2 to 9 silicon atoms;

hydrocarbon-based volatile oils, such as isoparaffins, isododecane and fluoro oils;

poly(C₁-C₂₀)alkylsiloxanes, such as those containing trimethylsilyl end groups, for example, linear polydimethylsiloxanes and alkylmethylpolysiloxanes such as cetyl dimethicone (CTFA name);

silicones modified with at least one group chosen from fluorinated and r non-fluorinated, aliphatic and aromatic groups, and functional groups, such as hydroxyl, thiol and amine groups;

phenylsilicone oils;

oils of animal, plant and mineral origin, such as animal and plant oils formed from fatty acid esters of polyols, for example, liquid triglycerides, such as sunflower oil, corn oil, soybean oil, marrow oil, grapeseed oil, sesame oil, hazelnut oil, apricot oil, almond oil, avocado oil, fish oils, glyceryl tricaprocaprylate, and plant and animal oils of formula R₁COOR₂, in which R₁ is a higher fatty acid residue containing from 7 to 19 carbon atoms and R₂ is a branched hydrocarbon-based chain containing from 3 to 20 carbon atoms, for example purcellin oil; liquid paraffin, liquid petroleum jelly, perhydrosqualene, wheat germ oil, beauty-leaf oil, sesame oil, macadamia oil, grapeseed oil, rapeseed oil, coconut oil, groundnut oil, palm oil, castor oil, jojoba oil, olive oil, cereal germ oil; fatty acid esters; alcohols; acetylglycerides; octanoates, decanoates and ricinoleates of alcohols and of polyalcohols; fatty acid triglycerides; glycerides;

fluoro oils and perfluoro oils;

silicone gums;

waxes of animal, plant, mineral and synthetic origin, such as microcrystalline waxes, paraffin, petrolatum, petroleum jelly, ozokerite wax, montan wax; beeswax, lanolin and its derivatives; candelilla wax, ouricury wax, carnauba wax, Japan wax, cocoa butter, cork fibre wax or sugar cane wax; hydrogenated oils that are solid at 25° C., ozokerites, fatty esters and glycerides that are solid at 25° C.; polyethylene waxes and the waxes obtained by Fischer-Tropsch synthesis; hydrogenated oils that are solid at 25° C.; lanolins; fatty esters that are solid at 25° C.; silicone waxes; fluoro waxes.

The compositions may also comprise any additive usually used in the envisaged field of application, such as surfactants, antioxidants, fragrances, essential oils, preserving agents, cosmetic and pharmaceutical active agents, vitamins, essential fatty acids, sphingolipids, self-tanning agents, sunscreens, film-forming polymers, thickeners, gelling agents, colorants, pigments, fillers and nacres.

A person skilled in the art can take care to select the optional additional compounds and the amount thereof, such that the advantageous properties of the composition according to the invention are not, or are not substantially, adversely affected by the envisaged addition.

The compositions of the invention can be in any pharmaceutical form normally used for topical application, such as in the form of aqueous, aqueous-alcoholic and oily solutions; oil-in-water, water-in-oil and multiple emulsions, of liquid and semi-liquid consistencies of the milk type, of soft, semi-solid and solid consistencies of the cream type; aqueous and oily gels; liquid, pasty and solid anhydrous products; aqueous and oily dispersions and dispersions in a solvent medium, of lotion and serum types; microemulsions; microcapsules; microparticles and vesicular dispersions of ionic and nonionic type; in fluid, thickened and gelled, semi-solid and soft paste forms; in solid forms, such as sticks and tubes.

The compositions may be more or less fluid and may have the appearance of white and coloured creams, ointments, milks, lotions, serums, pastes, and mousses. It can optionally be applied to skin in the form of an aerosol. It can also be in solid form, and for example in the form of a stick. It can be used as a care product and/or as a make-up product for skin.

When the composition of the invention is an emulsion, the proportion of the fatty phase can range from 5% to 80% by weight, such as from 5% to 50% by weight relative to the total weight of the composition. The fatty substances, the emulsifiers and the co-emulsifiers used in the composition in emulsion form can be chosen from those conventionally used in the field under consideration. The emulsifier and the co-emulsifier can be present in the composition in a proportion ranging from 0.3% to 30% by weight, such as from 0.5% to 20%, by weight relative to the total weight of the composition.

In addition, the tensioning agents used according to the invention may also be combined with other compounds known to those skilled in the art as tensioning agents having properties different from those of the agents used according to the invention, especially proteins and protein hydrolysates. As compounds of this type, mention may be made, for example, of milk proteins such as lactalbumin, plant proteins such as the soybean protein sold under the name Eleseryl by the company LSN and the oat derivative sold under the name “Reductine” by the company Silab, and nucleic acids such as DNA.

The compositions according to the invention can find a use especially as cosmetic or pharmaceutical compositions for skin, mucous membranes and/or semi-mucous membranes.

They can find use as products for protecting and caring for the skin of the face, the neck, the hands or the body, especially anti-wrinkle and anti-fatigue compositions for making skin look radiant. A use in make-up compositions for body and facial skin, such as lipsticks, foundations, tinted creams and concealer sticks; and antisun compositions and artificial tanning compositions, may also be envisaged.

The composition of the invention can be used as an anti-wrinkle composition for the skin of the face and/or the neck.

The invention is illustrated in greater detail in the examples, which follow.

### EXAMPLE 1

Preparation of the compound of formula (I) in which:

R₁═CH₃, X═O, R₂═—CH₂—C₆H₄-pOH, R₃═H and n=100 (theoretical index)

20 g (0.096 mol) of tyrosine N-carboxyanhydride, 0.51 g (0.003 mol) of sodium methoxide in methanol, and 200 ml of anhydrous tetrahydrofuran are introduced into a 500 ml round-bottomed flask with stirring.

The mixture is stirred vigorously for 6 hours at 60° C. A considerable evolution of CO₂ takes place. At the end of the reaction, 10 ml of water are added. The solvents are evaporated off under reduced pressure.

15 g of yellow powder are obtained, equivalent to a yield of 96%.

### EXAMPLE 2

Preparation of the compound of formula (I) in which:

R₁═H, X═O, R₂═H, R₃═CH₃, R′₂═—CH₂—C₆H₄-p-OH and R′₃═H.

1.34 g (6.5 mmol) of tyrosine N-carboxyanhydride, 0.083 g (0.72 mmol) of sarcosine N-carboxyanhydride, 10 ml of anhydrous dioxane and 0.043 ml of a 0.03% solution of triethanolamine in dioxane are introduced into a 25 ml round-bottomed flask with stirring. The mixture is heated at 37° C. for 24 hours. A considerable evolution of CO₂ takes place. The solvent is evaporated off under reduced pressure.

1.3 g of yellow powder are obtained.

The theoretical ratio of sarcosine units relative to the tyrosine units is 9.

### EXAMPLE 3

Preparation of the compound of formula (I) in which:

R₁═—(CH₂)₄—CH(NH₂)—COOH, X═NH, R₂═H, R₃═CH₃, R′₂═—CH₂—C₆H₄-p-OH and R′₃═H.

4.5 g (22.7 mmol) of tyrosine N-carboxyanhydride, 0.5 g (4.5 mmol) of sarcosine N-carboxyanhydride, 50 ml of anhydrous tetrahydrofuran and 1.5 ml (0.21 mmol) of a 1% solution of L-lysine in dioxane are introduced with vigorous stirring into a 100 ml round-bottomed flask equipped with a condenser and a bubbler.

The mixture is heated at the reflux point of the THF for 6 hours. A considerable evolution of CO₂ takes place. After cooling to 20° C., the whole is precipitated from 50 ml of ethyl acetate with stirring, and is then filtered through a No. 3 sinter funnel and dried under vacuum at 40° C.

3.7 g of white powder are obtained.

The theoretical ratio of tyrosine units relative to the sarcosine units is 5.

### EXAMPLE 4

### Anti-wrinkle Cream

A water-in-oil emulsion is prepared, comprising (% by weight):

### Phase A

Hydrogenated polyisobutene 5.5%

Isostearyl neopentanoate 3.5%

PEG-20 stearate 1%

Glyceryl stearate+PEG-100 stearate 2%

Cetyl alcohol 0.5%

Stearyl alcohol 0.5%

Stearic acid 1%

### Phase B

Cyclomethicone 11%

### Phase C

Polyacrylamide+C13-14 isoparaffin+Laureth-7 1%

### Phase D

Compound of Example 1 7%

Water 25%

### Phase E

Preserving agents qs

Triethanolamine 0.03%

Demineralized water qs 100%

Phase A is heated with stirring until homogeneous. After cooling, phase B is added. Phase E is heated with stirring, and E is then poured into A with continued stirring. After cooling to 50° C., phase C is incorporated into the emulsion, followed by phase D.

A water-in-oil emulsion that may be used as an anti-wrinkle cream is obtained.

### EXAMPLE 5

### Anti-ageing Serum

A serum is prepared by mixing together the following (% by weight):

Polyacrylamide+C13-14 isoparaffin+Laureth-7 1%

Xanthan gum 0.2%

PVM/MA decadiene crosspolymer 0.2%

Triethanolamine 0.2%

Compound of Example 1 3.5%

Preserving agents qs

Water qs 100%

### Example 6

### Evaluation of the Tensioning Effect by Measurement using a Dermometer

The “tensioning” effect of the compounds according to the invention is evaluated by measurement using a dermometer. This device was described by L. Rasseneur et al. in Influence des Différents Constituants de la Couche Cornée sur la Mesure de son Elasticité [Influence of the Various Constituents of the Horny Layer on the Measurement of its Elasticity], International Journal of Cosmetic Science, 4, 247-260 (1982). The principle consists in measuring, before treatment and after treatment, the length of a test sample of isolated stratum corneum and in determining the percentage of retraction of the test sample.

Test samples of 0.6 cm×0.4 cm of stratum corneum ranging from 10 to 20 μm in thickness are used, mounted on the MTT 610 extensiometer sold by the company Diastron.

The test sample is placed between two jaws and then left for 12 hours in an atmosphere at 30° C. and 40% relative humidity. These jaws are then attached to the dermometer.

The test sample is pulled at a speed of 1 mm/minute by a length of between 5% and 10% of the initial length to determine the length L₀ at and above which the test sample begins to exert a force on the jaws that is detected by the device.

The test sample is then relaxed, after which 2 mg of the test composition are applied over the entire surface of the stratum corneum.

To prepare these compositions, the compounds are dissolved in hot (60° C.) DMF, to a concentration of 7% by weight; the solution is maintained at 60° C. until completely dissolved. It is cooled to 30° C. before use. The composition is then applied to the test sample. After total evaporation of the composition (drying at room temperature for 30 minutes), the test sample is pulled under the same conditions as those described above so as to determine also the length L₁ for the treated test sample.

The percentage of retraction is determined by the ratio:

100×(L₁−L₀)/L₀.

To characterize a tensioning effect, this percentage must be negative, and the tensioning effect is proportionately greater the larger the absolute value of the percentage of retraction.

The following results are obtained (mean and standard deviation on 7 samples):

% Variation in the length of the sample of stratum corneum and kinetics of the effect:

| Compound | 1H | 2H | 3H | Std-1H | Std-2H | Std-3H |
|---|---|---|---|---|---|---|
| Example 1 | −0.9 | −1.6 | −1.5 | 1.4 | 1.1 | 1.1 |
| Example 2 | −1.3 | −1.1 | −0.9 | 0.7 | 0.7 | 0.5 |
| Example 3 | −1.5 | −1.1 | −0.5 | 1.4 | 1.0 | 1.1 |

The compositions can be selected to show effectiveness within the first hour and onward, e.g., Example 2, within the first two hours and onward, e.g., Example 1, or during the first two hours, e.g., Example 3.

What is claimed is:

1. An anti-wrinkle composition comprising, in a physiologically acceptable medium, at least one material chosen from polymers and salts thereof, of formula (I): 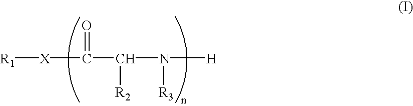
in which:
X is chosen from —O—, —S— and —NR, wherein R is chosen from hydrogen and linear and branched, saturated and unsaturated C₁-C₆ hydrocarbon-based radicals,
R₁ is chosen from:
(i) hydrogen,
(ii) linear and branched, saturated and unsaturated C₁-C₄₀ hydrocarbon-based radicals, optionally substituted with at least one group chosen from hydroxyl and —NR′R″, wherein R′ and R″ are chosen, independently of each other, from hydrogen and linear and branched, saturated and unsaturated C₁-C₆ hydrocarbon-based radicals; the C₁-C₄₀ hydrocarbon-based radicals being optionally interrupted with at least one hetero atom chosen from N, O and Si, and
(iii) a radical chosen from 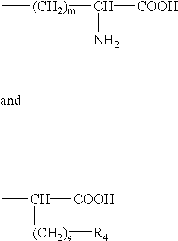
in which:
m is 1, 2, 3, 4 or 5;
s is an integer between 0 and 4 inclusive;
R₄ is chosen from hydrogen, —NH₂, —OH, —SH, —CHOHCH₃, —CONH₂, 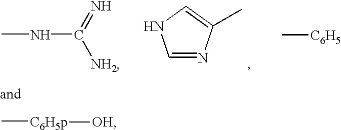
R₂ is chosen from hydrogen, linear and branched, saturated and unsaturated C₁-C₈ hydrocarbon-based radicals, and a radical chosen from —CH₂OH, —CHOH—CH₃, —CH₂C₆H₅, —CH₂C₆H₄p—OH and —(CH₂)ₜ—NH₂, with t being 1, 2, 3, 4 or 5,
R₃ is chosen from hydrogen, and linear and branched, saturated and unsaturated C₁-C₆ hydrocarbon-based radicals, and
n is an average number of repeating units of greater than 1, such that the weight average molecular weight of the at least one material is between 200 and 200 000 inclusive, the repeating unit being either identical for the same material, or different.

2. The composition of claim 1, wherein X is chosen from O, S and N—CH₃.

3. The composition of claim 1, wherein R₁ is chosen from hydrogen, linear and branched, saturated and unsaturated C₁-C₂₂ hydrocarbon-based radicals, and a radical chosen from:

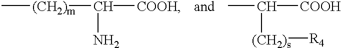

4. The composition of claim 3, wherein R₁ is a C₄-C₂₀ hydrocarbon-based radical.

5. The composition of claim 1, wherein R₂ is chosen from hydrogen, linear and branched, saturated and unsaturated C₁-C₆ hydrocarbon-based radicals, and —CH₂C₆H₄p-OH.

6. The composition of claim 1, wherein R₃ is chosen from hydrogen and linear and branched, saturated and unsaturated C₁-C₄ hydrocarbon-based radicals.

7. The composition of claim 6, wherein R₃ is chosen from methyl and ethyl radicals.

8. The composition of claim 1, wherein R₁ is chosen from:
C₁₅H₃₁—CH(OH)—CH(CH₂OH)—
C₁₀H₂₁—CH(C₈H₁₇)—CH₂—
C₁₆H₃₃—
C₈H₁₇—CH═CH—C₈H₁₆— 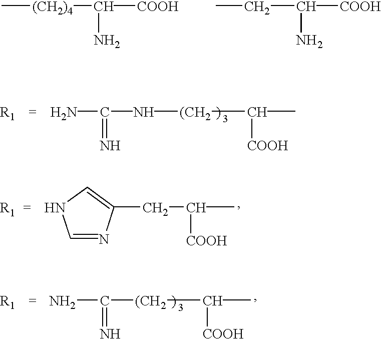
and
CH₂(OH)—CH(OH)—CH(OH)—CH(OH)—CH(OH)—CH₂—.

9. The composition of claim 1, wherein n is between 3 and 500 inclusive.

10. The composition of claim 1, wherein n is such that the weight average molecular weight of the at least one material is between 300 and 50 000 inclusive.

11. The composition of claim 1, wherein the at least one material is present, alone or as a mixture, in an amount of from 0.001% to 30% by weight relative to the total weight of the composition.

12. The composition of claim 11, wherein the at least one material is present, alone or as a mixture, in an amount of from 0.01% to 15% by weight relative to the total weight of the composition.

13. The composition of claim 1, wherein the physiologically acceptable medium comprises a cosmetically acceptable medium.

14. The composition of claim 1, wherein the physiologically acceptable medium comprises a pharmaceutically acceptable medium.

15. The composition of claim 1, further comprising tensioning agents chosen from proteins, protein hydrolysates, and nucleic acids.

16. The composition of claim 15, wherein the proteins are chosen from milk proteins, plant proteins, and oat derivatives.

17. The composition of claim 16, wherein the milk proteins comprise lactalbumin.

18. The composition of claim 16, wherein the plant proteins comprise soybean protein.

19. The composition of claim 15, wherein the nucleic acids comprise DNA.

20. The composition of claim 1, provided in a form chosen from: solutions chosen from aqueous, aqueous-alcoholic, and oily solutions; emulsions chosen from oil-in-water, water-in-oil, and multiple emulsions, the emulsions having a consistency chosen from liquid and semi-liquid milk consistencies, and soft, semi-solid and solid cream consistency; gels chosen from aqueous and oily gels; anhydrous products chosen from liquid, pasty and solid anhydrous products; lotion and serum dispersions chosen from aqueous dispersions, oily dispersions, and dispersions in a solvent medium; microemulsions; microcapsules; ionic and nonionic dispersions chosen from microparticles and vesicular dispersions; fluid, thickened, gelled, semi-solid, and soft paste forms; solid forms chosen from sticks and tubes; white creams, coloured creams, ointments, milks, lotions, serums, pastes, and mousses; and aerosols.

21. The composition of claim 1, provided in a form chosen from:
(i) products for protecting and caring for the skin of the face, the neck, the hands and the body;
(ii) make-up compositions for body and facial skin;
(iii) antisun compositions; and
(iv) artificial tanning compositions.

22. The composition of claim 21, wherein the products (i) are chosen from anti-wrinkle and anti-fatigue compositions, for making the skin look radiant.

23. The composition of claim 21, wherein the make-up compositions (ii) are chosen from lipsticks, foundations, tinted creams, and concealer sticks.

24. The composition of claim 1, provided in the form chosen from anti-wrinkle compositions for the skin of the face and the neck.

25. A method for reducing, fading out and/or smoothing out wrinkles and/or fine lines in skin, comprising:
applying to the skin a composition comprising, in a physiologically acceptable medium, at least one material chosen from polymers and salts thereof, of formula (I): 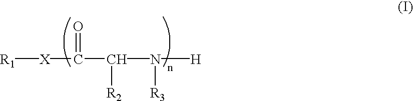
in which:
X is chosen from —O—, —S— and —NR, wherein R is chosen from hydrogen and linear and branched, saturated and unsaturated C₁-C₆ hydrocarbon-based radicals,
R₁ is chosen from:
(i) hydrogen,
(ii) linear and branched, saturated and unsaturated C₁-C₄₀ hydrocarbon-based radicals, optionally substituted with at least one group chosen from hydroxyl and —NR′R″, wherein R′ and R″ are chosen, independently of each other, from hydrogen, and linear and branched, saturated and unsaturated C₁-C6 hydrocarbon-based radicals; the C₁-C₄₀ hydrocarbon-based radicals being optionally interrupted with at least one hetero atom chosen from N, O and Si, and
(iii) a radical chosen from 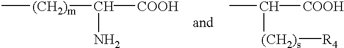
in which:
m is 1, 2, 3, 4 or 5;
s is an integer between 0 and 4 inclusive;
R₄ is chosen from hydrogen, —NH₂, —OH, —SH, —CHOHCH₃, —CONH₂, 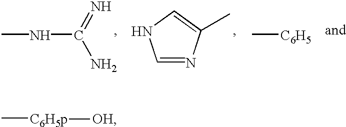
R₂ is chosen from hydrogen, linear and branched, saturated and unsaturated C₁-C₈ hydrocarbon-based radicals, and radicals chosen from —CH₂OH, —CHOH—CH₃, —CH₂C₆H₅, —CH₂C₆H₄p—OH and —(CH₂)ₜ—NH₂, with t being 1, 2, 3, 4 or 5,
R₃ is chosen from hydrogen and linear and branched, saturated and unsaturated C₁-C₆ hydrocarbon-based radicals, and
n is an average number of repeating units of greater than 1, such that the weight average molecular weight of the at least one material is between 200 and 200 000 inclusive, the repeating unit being either identical for the same material, or different.

26. A method of manufacturing a pharmaceutical composition for reducing, fading out and/or smoothing out wrinkles and/or fine lines in skin, comprising:
adding to a physiologically acceptable medium, at least one material chosen from polymers and salts thereof, of formula (I): 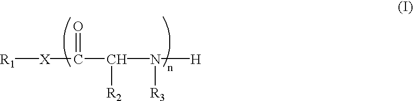
in which:
X is chosen from —O—, —S— and —NR, wherein R is chosen from hydrogen and linear and branched, saturated and unsaturated C₁-C₆ hydrocarbon-based radicals,
R₁ is chosen from:
(i) hydrogen,
(ii) linear and branched, saturated and unsaturated C₁-C₄₀ hydrocarbon-based radicals, optionally substituted with at least one group chosen from hydroxyl and —NR′R″, wherein R′ and R″ are chosen, independently of each other, from hydrogen and linear and branched, saturated and unsaturated C₁-C₆ hydrocarbon-based radicals; the C₁-C₄₀ hydrocarbon-based radicals being optionally interrupted with at least one hetero atom chosen from N, O and Si, and
(iii) a radical chosen from 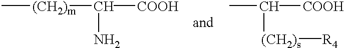
in which:
m is 1, 2, 3,4 or 5;
s is an integer between 0 and 4 inclusive;
R4 is chosen from hydrogen, —NH₂, —OH, —SH, —CHOHCH₃, —CONH₂, 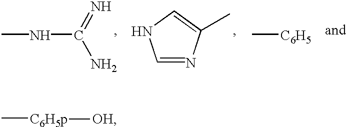
R₂ is chosen from hydrogen, linear and branched, saturated and unsaturated C₁-C₈ hydrocarbon-based radicals, and radicals chosen from —CH₂OH, —CHOH—CH₃, —CH₂C₆H₅, —CH₂C₆H₄p—OH and —(CH₂)ₜ—NH₂, with t being 1, 2, 3, 4 or 5,
R₃ is chosen from hydrogen and linear and branched, saturated and unsaturated C₁-C₆ hydrocarbon-based radicals, and
n is an average number of repeating units of greater than 1, such that the weight average molecular weight of the at least one material is between 200 and 200 000 inclusive, the repeating unit being either identical for the same material, or different.

27. A tensioning agent for reducing, smoothing out and/or fading out wrinkles and/or fine lines in skin, comprising, in a physiologically acceptable medium, at least one material chosen from polymers and salts thereof, of formula (I): 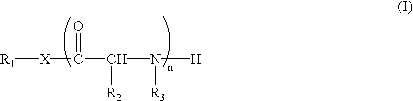
in which:
X is chosen from —O—, —S— and —NR, wherein R is chosen from hydrogen and linear and branched, saturated and unsaturated C₁-C₆ hydrocarbon-based radicals,
R₁ is chosen from:
(i) hydrogen,
(ii) linear and branched, saturated and unsaturated C₁-C₄₀ hydrocarbon-based radicals, optionally substituted with at least one group chosen from hydroxyl and —NR′R″, wherein R′ and R″ are chosen, independently of each other, from hydrogen and linear and branched, saturated and unsaturated C₁-C₆ hydrocarbon-based radicals; the C₁-C₄₀ hydrocarbon-based radicals being optionally interrupted with at least one hetero atom chosen from N, O and Si, and
(iii) a radical chosen from 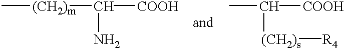
in which:
m is 1, 2, 3, 4 or 5;
s is an integer between 0 and 4 inclusive;
R₄ is chosen from hydrogen, —NH₂, —OH, —SH, —CHOHCH₃, —CONH₂, 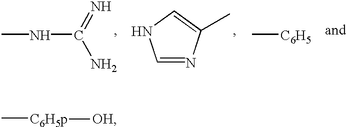
R₂ is chosen from hydrogen, linear and branched, saturated and unsaturated C₁-C₈ hydrocarbon-based radicals, and radicals chosen from —CH₂OH, —CHOH—CH₃, —CH₂C₆H₅, —CH₂C₆H₄p—OH and —(CH₂)ₜ—NH₂, with t being 1, 2, 3, 4 or 5,
R₃is chosen from hydrogen and linear and branched, saturated and unsaturated C₁-C₆ hydrocarbon-based radicals, and
n is an average number of repeating units of greater than 1, such that the weight average molecular weight of the at least one material is between 200 and 200 000 inclusive, the repeating unit being either identical for the same material, or different.
